(19) United States
(12) Patent Application Publication
(10) Pub. No.: US 2010/0113597 A1
(43) Pub. Date: May 6, 2010

(54) PHARMACEUTICAL COMPOSITION FOR PREVENTION OF PROGRESS OF INTESTINAL CONSTRICTION ASSOCIATED WITH CROHN'S DISEASE

(76) Inventor: Nobuhide Oshitani, Izumiotsu-shi (JP)

Correspondence Address: SUGHRUE MION, PLLC, 2100 PENNSYLVANIA AVENUE, N.W., SUITE 800, WASHINGTON, DC, 20037, US

(21) Appl. No.: 12/593,834

(22) PCT Filed: Mar. 26, 2008
(86) PCT No.: PCT/JP2008/055632

§ 371 Date: Sep. 29, 2009

(30) Foreign Application Priority Data
Mar. 30, 2007 (JP) 2007-089968

(51) Int. Cl. A61K 31/196; A61P 1/00; A61P 37/02

(52) U.S. Cl. 514/563

(57) ABSTRACT

The present invention provides an oral pharmaceutical composition for inhibiting the progression of intestinal stricture associated with Crohn's disease. That is, the present invention relates to a pharmaceutical composition for inhibiting the progression of intestinal stricture associated with Crohn's disease which comprises as an active ingredient N-(3,4-dimethoxycinnamoyl)anthranilic acid (generic name: tranilast) or a pharmaceutically acceptable salt thereof. The present invention can provide a pharmaceutical composition useful as an agent for inhibiting the progression of intestinal stricture associated with Crohn's disease for medical therapy.

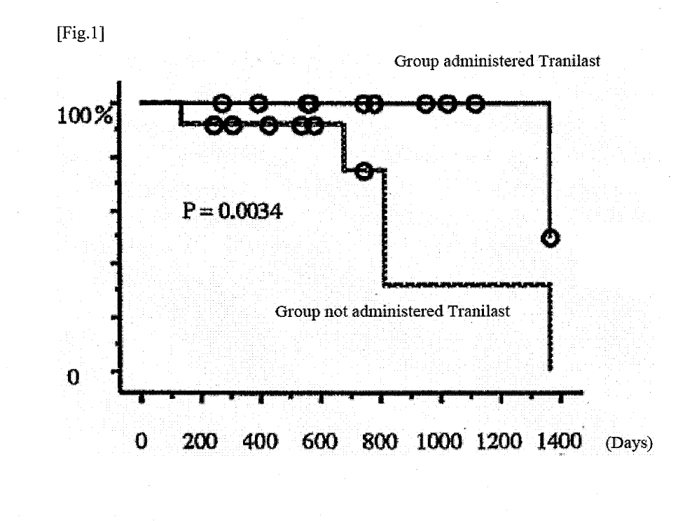

### TECHNICAL FIELD

The present invention relates to a pharmaceutical composition for inhibiting the progression of intestinal stricture associated with Crohn's disease which comprises as an active ingredient tranilast (chemical name: N-(3,4-dimethoxycinnamoyl)anthranilic acid) or a pharmaceutically acceptable salt thereof.

### BACKGROUND ART

Crohn's disease is one of non-specific inflammatory bowel diseases and a cryptogenic intractable long-standing chronic disease consisting of granulomatous inflammatory lesion associated with edema developed throughout the digestive tract and fibrotic ulcer, and any radical treatment has not been established.

The diagnosis criteria of Crohn's disease was significantly revised by a specified disease investigative research group of the Japanese Health and Welfare Ministry on intractable inflammatory intestinal tract disorders in 1996, and clinical observation findings became emphasized. In the criteria, three major criteria: A) longitudinal ulcer, B) cobblestone appearance, C) noncaseating epithelioid cell granuloma and two minor criteria: a) tandem irregular-shaped ulcer or aphtha, b) irregular-shaped ulcer or aphtha observed in both of the upper digestive tract and lower digestive tract are raised, and Crohn's disease is diagnosed based on these 5 items findings.

Ulcerative colitis is known as an intractable disease classified in the same non-specific inflammatory bowel diseases with Crohn's disease, but it has been found that their clinical presentations, pathological appearances, endoscopic images and X-ray images are completely different from each other based on accumulation of various research results, and different results were obtained from immune aspect as well. Therefore, their diagnostic criteria are different, and different treatment guidelines have been provided for each of them.

Treatments of Crohn's disease are basically supportive measures mainly including medical therapy, and the primary purpose is to suppress the above clinical symptoms, maintain the condition with decreased symptoms wherein the symptoms are stable, prevent relapse or exacerbation and enable the patient to return to society.

Medical therapy includes nutrition therapy and drug therapy. As agents for drug therapy, a 5-aminosalitylic acid preparation such as salazosulfapyridine and mesalazine; an adrenocorticosteroid such as prednisolone; an immunomodulator such as azathioprine and 6-mercaptopurine; an antimicrobial such as metronidazole and ciprofloxacin; a human anti-TNFα monoclonal antibody preparation such as infliximab; and the like are used.

When disorders can not be improved even by such medical therapies and are severe and a severe complication can not be improved, a surgery to remove the lesion area surgically is forced to be introduced. However, incidence of reoccurrence after surgeries is high, and surgery frequently is required again and again. That mostly ends up with short-bowel syndrome which requiring continuously intravenous hyperalimentation at high rates. Therefore, surgical treatment is done as a last resort, and its absolute application is limited to intestinal obstruction, perforation, large blood loss, toxic megacolon and associated cancer.

Crohn's disease is frequently associated with intestinal stricture, fistula, trephination, adhesion or the like, and especially, intestinal obstruction is an important issue in the treatment of Crohn's disease. The existing agents for medical therapy is only to suppress inflammatory symptoms, a main symptom of Crohn's disease, and maintain adequate nutritional status, but not to affect intestinal obstruction.

In a draft therapeutic guideline 2006 for intestinal stricture, trying endoscopical dilatation before surgical treatment was added. Such a physical dilatation may cause reoccurrence or restenosis at high rates similarly as surgeries, and furthermore, increase in severity followed by intestinal obstruction, which finally leads to surgical treatments. When a surgery is conducted, short-bowel syndrome can occur at high rates as mentioned above. Therefore, development of an agent for medical therapy to inhibit the progression of intestinal stricture associated with Crohn's disease has been desired.

Tranilast has inhibitory activities on chemical mediator release, excessive collagen synthesis and the like, and has been used widely as an agent for the treatment of allergic disorders such as allergic bronchial asthma, rhinitis, atopic dermatitis, allergic conjunctivitis and the like as well as the treatment of disorders associated with excessive collagen synthesis such as keloid, hypertrophic scar and the like. It has been also confirmed that tranilast causes less incidence of severe side effects even in a long-term use and is extremely safe. In addition, as a therapeutic agent comprising tranilast as an active ingredient, an agent for the prevention or treatment of a disease associated with excessive proliferation of vascular endothelial cells such as restenosis after percutaneous transluminal coronary angioplasty, arteriosclerosis and the like, an atherosclerosis inhibitor, an agent for prevention or treatment of chlamydial disorders, a neovascularization inhibitor, a corneal subepithelial opacity inhibitor, a secondary cataract inhibitor, an inhibitor of the progress of pterygium and postoperative recurrence of the same, an agent for the prevention or treatment of diseases associated with excessive proliferation of retinal pigment epithelial cells, an agent for the prevention or treatment of heart failure and the like have been reported (see Patent references 1 to 9).

Thus, it has been reported that tranilast exerts preventive and therapeutic effects for various diseases. However, there has been not any report about its effect on intestinal stricture in the above references, and it has not ever been reported or suggested that it is useful as an agent for inhibiting the progress of intestinal stricture associated with Crohn's disease. Furthermore, although the effects described in the above Patent references include a preventive or therapeutic effect of a disease associated with excessive proliferation of vascular smooth muscle cells such as restenosis after percutaneous transluminal coronary angioplasty, arteriosclerosis and the like, and an inhibitory effect on atherosclerosis, such vascular stenosis is a different disease from intestinal stricture of the present invention and they are clearly distinct. Therefore, these effects are completely different from each other and they are not analogical with each other.

On the other hand, it was reported that use of tranilast in combination with an adrenocorticosteroid or salazosulfapyridine was effective for decreasing the amount of the steroid, preventing exacerbation and maintaining the decreased symptoms in the treatment of ulcerative colitis which is classified as a nonspecific inflammatory bowel disease together with Crohn's disease (see Non-patent references 1 to 4). As mentioned above, however, although Crohn's disease is classified as a nonspecific inflammatory bowel disease together with ulcerative colitis, they are clearly distinct from each other from the viewpoints of clinical presentations, pathological appearances, endoscopic images and X-ray images and inflammation and different therapeutic guidelines are recommended for the treatment of each disease. In addition, while accompanying intestinal stricture is an important problem in Crohn's disease, ulcerative colitis is rarely accompanied with intestinal stricture. It is clear from this that it can not be predicted that something effective for the treatment of ulcerative colitis would be also effective for the inhibition of progress of intestinal stricture associated with Crohn's disease.

Furthermore, in the above Non-patent references 1 to 4, it is only described that the therapeutic effect of tranilast on ulcerative colitis is based on its stabilizing effect of mast cells by inhibiting mast cell degranulation and is caused by inhibiting exacerbation or getting worse of ulcerative colitis induced by chemical mediators such as histamine, serotonin and the like released from mast cells, and there is no description or suggestion that tranilast has an effect of delaying the progress of or inhibiting intestinal stricture associated with Crohn's disease.

Meanwhile, as a treated case of Crohn's disease, in a case administered tranilast instead of a steroid in addition to enteral nutrients and low residue diet, it was reported that the case was maintained without exacerbation of symptoms of Crohn's disease for 3 years (see Non-patent reference 5). In the case, however, tranilast was used for the purpose of anti-inflammation instead of a steroid due to side effects of the steroid. In addition, mepenzolate bromide as an antiflatulent was concomitantly used at the same time together with many general agents for the treatment of Crohn's disease such as the above-mentioned enteral nutrients and low residue diet, and so, it is unclear how tranilast was related to the effect on inhibition of exacerbation and maintenance of the decreased symptoms in the present case. Therefore, it can not be considered, of course, that the case showed the effect of tranilast alone.

In addition, in the above Non-patent reference 5, it is only described regarding the effect of tranilast that although its mechanism is uncertain, some relation of anti-allergic effect of tranilast is speculated because it is assumed that existence of some dietary antigen may be involved in causes and exacerbation factors of Crohn's disease. Thus, similarly to the above Non-patent references 1 to 4, the Non-patent reference 5 also does not disclose or suggest anything about that tranilast is useful as an agent for delaying the progress of or inhibiting intestinal stricture associated with Crohn's disease.

Patent reference 1: Japanese patent publication No. JPH06-135829 A

Patent reference 2: Japanese patent publication No. JPH09-227371 A

Patent reference 3: Japanese patent publication No. JPH10-139686 A

Patent reference 4: International publication No. WO97/29744 pamphlet

Patent reference 5: International publication No. WO98/13038 pamphlet

Patent reference 6: International publication No. WO98/16214 pamphlet

Patent reference 7: International publication No. WO98/35668 pamphlet

Patent reference 8: International publication No. WO98/47504 pamphlet

Patent reference 9: Japanese patent publication No. JP2001-64202 A

Non-patent reference 1: Mikio Inoue, Tomonori Minota, Rinsho Shokaki Naika (Clinical gastroenterological medicine), 1989, Vol. 4, pp. 1527-1534

Non-patent reference 2: Takayuki Matsumoto et. al., Shinsatsu-to-Shinyaku (Physical examination and New medicine), 1985, Vol. 22, pp. 657-666

Non-patent reference 3: Yoshikazu Yonei et. al., Shinsatsu-to-Shinyaku (Physical examination and New medicine), 1985, Vol. 22, pp. 1755-1761

Non-patent reference 4: Kazuya Makiyama et. al., Daicho Komon-shi (Journal of Colon and Anus), 1988, Vol. 41, pp. 819-825

Non-patent reference 5: Takeshi Kikuchi et. al., Nihon Shokakibyo Gakkai Zasshi (Japanese Journal of Gastroenterology Society), 1997, Vol. 94, No. 3, pp. 195-199

### DISCLOSURE OF THE INVENTION

### Objects to be Solved by the Invention

The present invention aims to provide a pharmaceutical composition useful as an agent for inhibiting the progression of intestinal stricture associated with Crohn's disease that has been difficult to achieve by existing agents for medical therapy.

### Means for Solving the Problems

The present inventors have studied earnestly to find an agent effective against the progression of intestinal stricture associated with Crohn's disease that has been difficult to achieve by agents for nutrition therapy and drug therapy of the existing agents for medical therapy. As a result, it was found tranilast significantly delayed or inhibited the progression of intestinal stricture associated with Crohn's disease, and a pharmaceutical composition comprising as an active ingredient tranilast or a pharmaceutically acceptable salt thereof are extremely useful as an agent for inhibiting the progression of intestinal stricture associated with Crohn's disease, thereby forming the basis of the present invention.

That is, the present inventors found that by repeated administration of tranilast 600 mg per day (200 mg three times a day) to patients with intestinal stricture but without any symptoms, the incidence of intestinal stricture symptoms caused by intestinal stricture was significantly inhibited in comparison with a group without administration of tranilast. The present invention was formed thereby.

The present invention relates to a pharmaceutical for delaying or inhibiting the progression of intestinal stricture associated with Crohn's disease. More particularly, the present invention relates to:

[1] a pharmaceutical composition for inhibiting the progression of intestinal stricture associated with Crohn's disease, which comprises as an active ingredient tranilast or a pharmaceutically acceptable salt thereof;

[2] a pharmaceutical composition as described in the above [1], which is an oral formulation;

[3] a pharmaceutical composition as described in the above [1] or [2], which comprises an amount of tranilast or a pharmaceutically acceptable salt thereof corresponding to 150 mg to 900 mg of tranilast as a daily dosage;

[4] a pharmaceutical composition as described in the above [3], which comprises an amount of tranilast or a pharmaceutically acceptable salt thereof corresponding to 300 mg to 600 mg of tranilast as a daily dosage;

[5] a pharmaceutical composition as described in the above [4], which comprises orally administering 200 mg three times daily; and the like.

### Effects of the Invention

The present invention can provide a pharmaceutical composition useful as an agent for inhibiting the progression of intestinal stricture associated with Crohn's disease for medical therapy.

### BRIEF DESCRIPTION OF DRAWINGS

The horizontal axis shows the transitional period from the beginning of the study (day) and the vertical axis shows the cumulative non-symptomatic stricture rate, respectively. The solid line and dashed line show the data of the group administered tranilast and the group not administered tranilast, respectively.

### BEST MODE TO PUT THE INVENTION TO PRACTICE

In the pharmaceutical composition of the present invention, inhibiting the progression of intestinal stricture associated with Crohn's disease includes delaying the progression of intestinal stricture associated with Crohn's disease, preventing intestinal obstruction, preventing or reducing the occurrence of symptoms associated with intestinal stricture or obstruction or the like.

Tranilast or a pharmaceutically acceptable salt thereof of the active ingredient of the pharmaceutical composition of the present invention can be prepared easily according to a known method or similar methods thereof.

As pharmaceutically acceptable salts of tranilast, for example, a salt with an inorganic base such as a sodium salt, a potassium salt, a calcium salt and the like; a salt with an organic amine or an amino acid such as morpholine, piperazine, pyrrolidine and the like can be illustrated.

When the pharmaceutical compositions of the present invention are employed in the practical treatment, various dosage forms are used depending on their usage. As examples of the dosage forms, oral formulations such as powders, granules, fine granules, dry syrups, tablets, capsules and the like are illustrated, and oral formulations are preferable.

The pharmaceutical compositions of the present invention can be prepared by suitably admixing with or by diluting and dissolving with an appropriate pharmaceutical additive such as excipients, disintegrators, binders, lubricants, diluents, buffers, isotonicities, antiseptics, moistening agents, emulsifiers, dispersing agents, stabilizing agents, dissolving aids and the like, and formulating the mixture in accordance with conventional methods according to procedures pharmaceutically used depending on the dosage forms. In the case of the uses in combination with other drug(s), they can be prepared by formulating each active ingredient together or individually in a similar manner as defined above.

For example, tablets can be formulated by, if desired, adding appropriate excipients, disintegrators, binders, lubricants and the like to tranilast or a salt thereof, and compressing the mixture in accordance with conventional methods. The tablets, further if desired, can be also coated to provide film-coated tablets, sugar-coated tablets, enteric-coated tablets and the like.

For example, capsules can be formulated by, if desired, adding appropriate excipients, lubricants and the like to tranilast, admixing, and then filling the compositions in appropriate capsules, or optionally formulating granules or fine-powders in accordance with conventional methods before filling.

Furthermore, the pharmaceutical composition of the present invention can be also used in combination with other agent(s) for nutrition therapy and/or drug therapy which are used for the treatment of Crohn's disease, and optionally with other anti-inflammatory agent, analgesic, antiulcer agent or the like. As the other agents for nutrition therapy, ELENTAL (registered trade mark) of a component nutrient, Enterued (registered trade mark) of a digestible nutrient, Twinline (registered trade mark) and the like can be illustrated. As the other agents for drug therapy, a 5-aminosalitylic acid preparation such as salazosulfapyridine and mesalazine; an adrenocorticosteroid such as prednisolone; an immunomodulator such as azathioprine and 6-mercaptopurine; an antimicrobial such as metronidazole and ciprofloxacin; a human anti-TNFa monoclonal antibody preparation such as infliximab; and the like can be illustrated.

In the case of uses of the pharmaceutical composition of the present invention in combination with the above one or more other drugs, the present invention includes either dosage forms of simultaneous administration as a single preparation or separated preparations in way of the same or different administration route, and administration at different dosage intervals as separated preparations in way of the same or different administration route.

When the pharmaceutical compositions of the present invention are employed in the practical treatment, the dosage of tranilast or a pharmaceutically acceptable salt thereof as the active ingredient is appropriately decided depending on the body weight, age, sex and degree of diseases of each patient, and an amount of tranilast or a pharmaceutically acceptable salt thereof is administered corresponding to approximately within the range of from 150 to 900 mg per day, preferably approximately within the range of from 300 to 600 mg per day as tranilast.

The dosing method is, for example, orally administering 1 to 3 times daily. In addition, in the case of the uses in combination with the above other drug(s), the dosage of the compound of the present invention can be decreased depending on the dosage of the other drug(s). The dosing period starts at the time of diagnosis of Crohn's disease, or anytime in case that the treatment of Crohn's disease has been already begun, particularly, in case that surgery or endoscopical dilatation is applied, before such a procedure, and preferably continues as long as adverse events do not occur. In addition, it is preferable to continuously administer as long as the intestinal stricture exists even in a condition with no subjective symptoms or decreased symptoms.

### Examples

The present invention is further illustrated in more detail by way of the following Examples and Comparative Examples. However, the present invention is not limited thereto.

### Example 1

### Tranilast's Effects on Inhibition of Progression of Intestinal Stricture Associated with Crohn's Disease

In 24 Crohn's disease patients with non-symptomatic intestinal stricture, the effect by tranilast on the progression of intestinal stricture associated with Crohn's disease was evaluated and compared between one group administered tranilast and another group not administered tranilast.

Test method
1) Background of Patient

The backgrounds of the 24 patients evaluated are shown in Table 1. There was no significant difference in backgrounds of the patients between the groups.

TABLE 1

| | | Group |
|---|---|---|
| | Group administered | not administered |
| | tranilast | tranilast |
| Items | (12 cases) | (12 cases) |
| Average age (years) | 35.0 | 37.5 |
| Gender rate (Male/Female) | 7/5 | 9/3 |
| Average disease | 7.3 | 11.5 |
| duration (years) | | |
| Location of the disease | | |
| Ileum | 3 | 7 |
| Colon | 4 | 0 |
| Ileocolon | 5 | 5 |
| Behavior of the disease | | |
| Stricturing | 7 | 4 |
| Penetrating | 5 | 5 |
| Location of stricture | | |
| Ileum | 7 | 8 |
| Colon | 5 | 4 |

2) Dosing Regimen

The 24 patients were randomly divided into two groups consisting of a group administered tranilast (12 cases) and a group not administered tranilast (12 cases). The patients of the group administered tranilast received tranilast 200 mg three times daily after each meal, and the patients of the group not administered tranilast did not received tranilast.

3) Endpoint

The primary endpoint was a cumulative non-symptomatic stricture rate which was a rate of cases wherein severe symptomatic intestinal stricture requiring balloon dilatation or surgery occurred. In addition, the diameters of the stricture at the beginning of observation (basal diameter) and at the end of observation (final diameter) were measured. The secondary endpoint was a monthly progression rate of the stricture obtained from the rate of the final diameter relative to the basal diameter.

### Results

1) Cumulative Non-Symptomatic Stricture Rate

The numbers of the cases who required balloon dilatation due to severe intestinal stricture were 1 case in the group administered tranilast and 5 cases in the group not administered tranilast, respectively. It was confirmed that the incidence of intestinal obstruction symptoms due to intestinal stricture was significantly low in the group administered tranilast from the result of the cumulative non-symptomatic stricture rate (p=0.0034).
2) Diameter of Stricture

The average diameters of the stricture at the beginning of observation (basal diameter) were 6.40 mm in the group administered tranilast and 6.35 mm in the group not administered tranilast, respectively. The average diameters of the stricture at the end of observation (final diameter) were 5.60 mm in the group administered tranilast and 5.05 mm in the group not administered tranilast, respectively. In addition, the monthly progression rate of the stricture (%) were 0.48% in the group administered tranilast and −0.86% in the group not administered tranilast, respectively. While tendency reducing the diameter of the stricture was observed in the group not administered tranilast, the reduction of the diameter of the stricture was smaller in the group administered tranilast.
3) Others

The median observation period were 782 days in the group administered tranilast and 559 days in the group not administered tranilast, respectively. In addition, 1 case in 12 cases of the group administered tranilast withdrew due to discontinued administration because of a reduced peripheral white blood cell count. In addition, during the observation period, 1 case in the group administered tranilast and 2 cases in the group not administered tranilast received infliximab infusion, 2 cases in the group administered tranilast and 1 case in the group not administered tranilast received oral prednisolone and 6 cases in the group administered tranilast and 7 cases in the group not administered tranilast received immunomodulators (azathioprine or 6-mercaptopurine), respectively. There were no particular influence caused by these drug administrations.

As mentioned above, it was demonstrated that the pharmaceutical composition of the present invention is extremely useful as an agent for inhibiting the progression of intestinal stricture associated with Crohn's disease.

### INDUSTRIAL APPLICABILITY

The pharmaceutical composition of the present invention is useful as an agent for inhibiting the progression of intestinal stricture associated with Crohn's disease.

1-5. (canceled)

6. A method for inhibiting the progression of intestinal stricture associated with Crohn's disease, which comprises administering an effective amount of tranilast or a pharmaceutically acceptable salt thereof.

7. A method as claimed in claim 6, which comprises administering in an oral formulation.

8. A method as claimed in claim 6 or 7, which comprises administering an amount of tranilast or a pharmaceutically acceptable salt thereof corresponding to 150 mg to 900 mg of tranilast as a daily dosage.

9. A method as claimed in claim 8, which comprises administering an amount of tranilast or a pharmaceutically acceptable salt thereof corresponding to 300 mg to 600 mg of tranilast as a daily dosage.

10. A method as claimed in claim 9, which comprises orally administering an amount of tranilast or a pharmaceutically acceptable salt thereof corresponding to 200 mg of tranilast three times daily.
